(19) United States
(12) Patent Application Publication
(10) Pub. No.: US 2007/0207107 A1
(43) Pub. Date: Sep. 6, 2007

(54) Silicone based emulsions for topical drug delivery

(76) Inventors: Gareth Winckle, Petaluma, CA (US); David W. Osborne, Santa Rosa, CA (US); Gordon J. Dow, Santa Rosa, CA (US)

Correspondence Address: HOWARD EISENBERG, ESQ., 1220 LIMBERLOST LANE, GLADWYNE, PA, 19035, US

(21) Appl. No.: 11/699,906

(22) Filed: Jan. 30, 2007

Related U.S. Application Data
(60) Provisional application No. 60/778,825, filed on Mar. 3, 2006.

(51) Int. Cl. A61K 8/89

(52) U.S. Cl. 424/70.12

(57) ABSTRACT

A water-in-oil emulsion is provided in which the lipophilic phase of the emulsion contains a silicone fluid and an emulsifier, a hydrophilic phase, and a pharmaceutically active compound. The active pharmaceutical ingredient is dissolved or dispersed in the emulsion and is partitioned in the emulsion so that all or a portion of the amount of the chemical compound dissolved or dispersed in the emulsion is dissolved or dispersed in the aqueous phase of the emulsion. The emulsion of the invention provides increased penetration into skin of the chemical compound dissolved or dispersed in the aqueous phase.

This application claims priority from pending U.S. Provisional Patent Application No. 60/778,825, filed on Mar. 3, 2006, which provisional patent application is incorporated herein in its entirety.

### FIELD OF THE INVENTION

The invention pertains to the field of emulsions for topical administration of active pharmaceutical ingredients (API) to the skin.

### BACKGROUND OF THE INVENTION

For the treatment of many dermatologic conditions, it is desirable to topically administer a medication that penetrates into the skin. However, the stratum corneum (SC) layer of the skin provides a significant barrier to the penetration of drugs based on molecular weight or molecular volume and degree of lipophilicity. Lipophilic drugs tend to be absorbed more readily into the skin as the intercellular lipid pathway is generally considered to be the primary route of SC penetration. In contrast, penetration of hydrophilic drugs into the skin is limited.

An alternative penetration pathway which bypasses the SC is the appendageal route. Epidermal appendages include hair follicles, sweat glands, and sebaceous glands. There is growing evidence that appendageal transport has been underestimated and may be of importance for a large range of chemical substances. One barrier to appendageal penetration of certain drugs is the presence of sebaceous lipids in the sebum produced by sebaceous glands. These lipids do not significantly interfere with penetration of lipophilic molecules but they present a significant barrier to penetration of hydrophilic molecules.

Silicones are a class of compounds based on polydialkylsiloxanes. These compounds are typically linear or cyclic polymers which are liquid at room temperature. The silicones are highly lipophilic and are miscible in most organic solvents but are non-miscible in water. The silicone compounds have been used in a variety of pharmaceutical and cosmetic formulations. They are accepted generally as being non-toxic and as having a negligible impact on the environment. Silicones commonly used in pharmaceutical formulations include the non-volatile silicone dimethicone, the volatile silicone cyclomethicone, simethicone (a blend of dimethicone and silicon dioxide), and the highly volatile silicones hexamethyldisiloxane and octamethyltrisiloxane.

Silicones have been used as excipients for pharmaceutical topical formulations. Dow Coming Corporation (Midland, Mich.) has introduced a range of products in their DOW CORNING® SILKY TOUCH line, as explained in their brochure Sene, et al, “Silicones as Excipients for Topical Pharmaceutical Applications: The Silky Touch Product Family from Dow Coming”. The Silky Touch line of products is a series of various silicones that are useful in topical formulations for the cosmetic and pharmaceutical industries.

Sene et al discloses that certain drugs, when formulated in silicone, exhibit differing penetration profiles into the deeper layers of the skin. Formulations containing one of three different drugs, ibuprofen, econazole, or hydrocortisone, were dissolved in 98% hexamethyldisiloxane and 2% silicone gum and were tested and compared to the same drugs in a silicone-free emulsion. Each of the drugs tested was dissolved in the silicone. It was found that the silicone increased the penetration of the ibuprofen into the deeper layers of the skin. However, the silicone formulation resulted in reduced penetration of econazole nitrate through the skin. With hydrocortisone, the silicone formulation resulted in the formation of a reservoir of hydrocortisone in the stratum corneum.

Thus, the results of the Sene study showed that the skin permeability of a drug dissolved in silicone was dependent on the particular drug used and that the skin penetration of any particular drug might be increased, decreased, or unchanged by dissolving the drug in silicone.

Each of the drugs tested in the Sene study was dissolved in silicone. However, Sene study did not address the skin penetration of a drug which is not dissolved in silicone. Many drugs that are useful for treating or preventing disorders of the skin are not soluble in silicone. A significant need exists for a formulation for topical administration to the skin that enables silicone-insoluble compounds, such as active pharmaceutical ingredients, to penetrate the stratum corneum and reach the epidermis and dermis.

### BRIEF DESCRIPTION OF THE DRAWINGS

FIG. 1 is a bar chart and a line chart showing tissue distribution and penetration data in dermatomed skin for Formulations 1.1, 1.2, and 1.4 containing the API verapamil HCl (n=6, mean). The bar chart (FIG. 1A) compares the three formulations in terms of tissue distribution of the API in epidermis, dermis, and receptor medium beneath the dermis. The line chart (FIG. 1B) shows percutaneous penetration of the API from the three formulations measured in the receptor medium.

FIG. 2 is a bar chart and a line chart showing tissue distribution and penetration data in dermatomed skin for Formulations 1.1, 1.3, and 1.5 containing the API verapamil HCl (n=6, mean). The bar chart (FIG. 2A) compares the three formulations in terms of tissue distribution of the API in epidermis, dermis, and receptor medium beneath the dermis. The line chart (FIG. 2B) shows percutaneous penetration of the API from the three formulations measured in the receptor medium.

FIG. 3 is a bar chart showing % of the dose applied of an oligonucleotide from successive tape strips following application of Formulations 2.5, 2.7, and 2.9 to dermatomed skin.

FIG. 4 is a bar chart showing % of the dose applied of an oligonucleotide in epidermis and dermis following application of Formulations 2.5, 2.7, and 2.9 to dermatomed skin.

FIG. 5 is a bar chart showing % of the applied dose of ¹⁴C-Caffeine recovered from the epidermis and dermis following application of Formulations 2227-60, 2227-65, 2227-70, 2227-71, 2227-78, 2227-84, 2227-85, 2227-91, 2227-32 and 2227-33A-34 to dermatomed skin.

FIG. 6 is a bar chart showing % of the applied dose of ¹⁴C-Glucose recovered from the epidermis and dermis following application of Formulations 2227-60, 2227-70, 2227-71, 2227-84, 2227-85, 2227-32 and 2227-33A-34 to dermatomed skin.

### DESCRIPTION OF THE INVENTION

It has been discovered that silicone fluid is capable of promoting penetration into skin of a chemical compound that is dissolved or dispersed, in whole or in part, in the hydrophilic phase of a water-in-oil emulsion. The discovery of the invention is especially surprising in that the silicone fluid of the emulsion has been found to promote the penetration of both hydrophilic and hydrophobic chemical compounds, so long as the chemical compound is dissolved or dispersed to some extent in the hydrophilic phase of the emulsion. Thus, combining a chemical compound, such as an active pharmaceutical ingredient (API), in a silicone-based water-in-oil emulsion system provides for enhanced delivery of the chemical compound into the tissues of the skin beyond the stratum corneum barrier compared with similar emulsion systems lacking silicone.

In one embodiment, the invention is a water-in-oil emulsion that contains a hydrophilic, such as aqueous, internal phase in which is dissolved or dispersed a chemical compound, such as an API, in a continuous, also referred to an external, silicone phase containing an emulsifier.

In another embodiment, the invention is a method for making a water-in-oil emulsion containing a silicone fluid. According to this embodiment, a hydrophilic solvent such as water is combined with a silicone oil and an emulsifier to produce a stable water-in-oil emulsion. A chemical compound, such as an API, is dissolved or dispersed in the hydrophilic solvent either before the hydrophilic solvent is combined with the silicone oil and the emulsifier or after the water-in-oil emulsion has been formed.

In another embodiment, the invention is a formulation for topical administration to the skin of a mammalian subject in need thereof which formulation is an emulsion as described above containing an aqueous internal phase, a chemical compound, such as a small molecule or a polymer, dissolved or dispersed in whole or in part in the hydrophilic solvent phase, and a continuous silicone phase. The chemical compound preferably is an API.

In another embodiment, the invention is a method for increasing the penetration of a chemical compound through the stratum corneum of mammalian skin. According to this embodiment of the invention, an emulsion is provided that contains a hydrophilic internal phase, such as a water-based internal phase, and a chemical compound dissolved or dispersed in whole or in part in the hydrophilic solvent and a silicone lipophilic phase containing an emulsifier, such as a silicone-based surfactant, and the emulsion is administered topically to the skin of the mammal.

It is conceived that the emulsion of the invention provides increased penetration of chemical compounds dissolved or dispersed, in whole or in part, in the inner hydrophilic aqueous phase of the emulsion because the continuous silicone phase dissolves and/or fluidizes sebaceous lipids and allows the delivery of hydrophilic aqueous phase droplets into and through the sebaceous lipid barrier. This enables the delivery of molecules dissolved or dispersed in the aqueous phase, such as drugs, to the SC surfaces of the hair canal and deeper into the pilosebaceous unit where the effectiveness of the skin barrier is reduced.

The silicone of the emulsion is a fluid at room temperature and may also be referred to as a silicone fluid or as a silicone oil. The silicone may be a volatile or non-volatile silicone or a combination of volatile and non-volatile silicones. Any silicone fluid is suitable for the emulsion of the invention. However, silicone fluids that are extremely volatile, such as hexamethyldisiloxane and octamethyltrisiloxane, are not preferred because, upon application to the surface of the skin, they typically provide insufficient residence time on the skin surface and so are generally ineffective in promoting the penetration of the compound into the skin. For example, the emulsion of the invention, however, may contain one or more of these extremely volatile silicones as an additional optional component in combination with one or more other silicones that are less volatile so long as the concentration of the less volatile silicones in the emulsion is sufficient to promote the penetration of a chemical compound into the skin. Examples of preferred silicone fluids include dimethicone (non-volatile) and cyclomethicone (volatile). Examples of additional suitable silicone fluids are cyclopentasiloxane, polydimethylsiloxane, cyclotetrasiloxane, decylmethylcyclopentasiloxane, and octylmethylcyclotetrasiloxane.

The concentration of the silicone in the emulsion is that which, by itself or in combination with other lipophilic solvents present in the emulsion, is sufficient to form a stable water-in-oil emulsion and which concentration of silicone promotes penetration of a chemical compound dissolved in the aqueous phase of the emulsion into skin. Preferably but not necessarily, silicone oil is the major component of the external phase of the emulsion. *

The lipophilic silicone phase of the emulsion contains an emulsifier, such as a surfactant, in a concentration sufficient to form a stable water-in-oil emulsion. A stable emulsion, as defined herein, is one in which no phase separation is visible upon unaided visual inspection when stored at room temperature for one month. Preferably, the emulsion is sufficiently stable so that no phase separation is visible for a period longer than one month, for example for one to 24 months, or longer. Preferably, the emulsifier is a silicone-based surfactant. Examples of silicone-based surfactants that are suitable as the emulsifier in the lipophilic phase of the emulsion of the invention include PEG-11 methyl ether dimethicone, PEG-10 dimethicone, laurylmethicone copolyol, stearoxymethicone/dimethicone copolymer, alkylmethyl siloxane copolymer such as Silky Touch Emulsifier 10 (Dow Corning Corporation), and cyclomethicone/dimethicone copolyol, such as DC 3225C Formulation Aid (ICNI name:) marketed by Dow Coming Corporation.

The concentration of the emulsifier in the emulsion of the invention is typically in the range of 1% to 15% w/w or higher. Preferably, the concentration of the emulsifier is between 2% to 8% w/w of the total emulsion.

The silicone phase mav include additional components or additives, if desired. Such additional components or additives may include one or more hydrocarbon-based waxes or oils. Examples of such waxes or oils include, but are not limited to, squalane, dibutyl sebacate, light mineral oil, mineral oil, isopropyl laurate, isopropyl myristate, isopropyl palmitate, isopropyl strearate, octyl palmitate, myristyl alcohol, oleyl alcohol, oleic acid, myristyl lactate, diisopropyl adipate, octyldodecanol, caproic acid, caprylic acid, capric acid, capric/caprylic triglycerides, glyceryl trioctante, C12-15 alkyl benzoate, benzyl benzoate, tridecyl neopentanoate, castor oil, spermaceti, petrolatum, and alpha terpineol. Other pharmaceutically or cosmetically acceptable or preferred constituents may also be included, if desired.

The hydrophilic phase of the emulsion generally, but not necessarily, contains water as a solvent in which the chemical compound is dissolved. In addition to or in place of water, the solvent of the hydrophilic phase may be any hydrophilic solvent that is suitable for topical application to the skin, is miscible with water and immiscible with the external silicone phase of the emulsion, and is capable of solubilizing or dispersing the chemical compound of the emulsion. Examples of suitable hydrophilic solvents that may be used in addition to water or in place of water include ethanol; polyethylene glycol; glycerin; propylene glycol; diethylene glycol monoethyl ether; hexylene glycol; 3-propanediol; 1,2-butanediol; 1,2,3-propanetriol; 1,3-butanediol; 1,4-butanediol; 2,3-butanediol; 1,2,6-hexanetriol; benzyl alcohol; isopropyl alcohol; and 2-amino-2-methyl-1-propanol.

The concentration of the hydrophilic solvent such as water in the emulsion of the invention is that which is sufficient to solubilize or disperse all or a portion of the chemical compound, such as the API, of the emulsion. Conceivably, there is no lower limit for the concentration of the hydrophilic solvent so long as an emulsion is obtained. An example is if the chemical compound of the emulsion is present in a very low concentration. An additional example is if the chemical compound itself is sufficiently hydrophilic and is a liquid at room temperature so that it by itself can constitute the hydrophilic phase or be a major component of the hydrophilic phase. As a practical matter, however, the emulsion typically requires a minimum of about 1% to 3% water or other hydrophilic solvent in order to dissolve or disperse sufficient quantities of the chemical compolmd to be therapeutically beneficial. The upper limit of the concentration of water, or other hydrophilic solvent, depends primarily on the stability of the water-in-oil emulsion that may be obtained. Generally, a concentration of hydrophilic phase higher than 50% favors the formation of an oil-in-water emulsion. However. it is well known in the art to make stable water-in-oil emulsions with an internal hydrophilic phase of up to about 80%. Therefore, depending on such factors including skin tolerance (for hydrophilic solvents other than water), solubility of API, and emulsion stability, the concentration of water and/or other hydrophilic solvent may be as high as 80%.

The chemical compound of the emulsion is one for which it is desired to have increased penetration into the skin. Typically, the compound is a pharmaceutically active compound such as a drug, often referred to as an API, that is useful in the treatment or prophylaxis of a dermatologic condition. In a preferred embodiment, the chemical compound of the emulsion is a hydrophilic compound that is dissolved exclusively in the inner hydrophilic phase, and which is insoluble or essentially insoluble in the lipophilic silicone continuous phase, referred to herein as being dissolved essentially exclusively in the hydrophilic phase. The chemical compound of the emulsion may also be one that is partitioned between both the hydrophilic and lipophilic phases of the emulsion. Alternatively, the compound may be one that is highly lipophilic and that is partitioned almost exclusively in the external oil phase. Such lipophilic compounds that are suitable for the invention are those that are partitioned to some degree in the hydrophilic as well as in the lipophilic phase. That is, compounds that are suitable for the invention are those that are dissolved, to some extent, in the aqueous phase. It is conceived that the emulsion and method of the invention increases the penetration of the portion of a chemical compound dissolved in an emulsion that is dissolved within the aqueous phase of the emulsion. It is preferable, although not essential, that the solubility of an API in the inner hydrophilic phase of the emulsion of the invention is sufficiently high so that an effective therapeutic concentration of the API in the emulsion can be obtained. For example, if an API is partitioned in an emulsion of the invention predominately in the lipophilic phase, the delivery of the API into skin will be augmented by the emulsion and method of the invention by increasing the penetration of the portion of the API that is partitioned within the aqueous phase of the emulsion. Thus, any chemical compound is suitable for the invention that can be partitioned in the emulsion of the invention at a ratio that is less than 100% in the lipophilic phase of the emulsion.

As another example, the chemical compound, such as an API, of the emulsion may be one that is hydrophobic and that is dispersed within the hydrophilic phase of a water-in-oil microsuspension. Such a microsuspension is included within the scope of the emulsion of the invention. The chemical compound may or may not be soluble to some extent within the silicone of the oil phase of the emulsion.

Optionally, the emulsion of the invention may contain an additional API, which API may be dissolved or dispersed in whole or in part in the hydrophilic phase and/or in the lipophilic phase of the emulsion. The additional API may be one of which the penetration into skin is increased by the method and/or composition of the invention. Alternatively, the additional API may be one of which the penetration into skin is not increased by the method and/or composition of the invention.

A co-emulsifier is an optional component that is preferably included in the silicone-containing emulsion of the invention. The co-emulsifier facilitates the formation of a physically stable water-in-silicone emulsion, enhances the physical stability of the emulsion, and may enhance penetration of the emulsion into the viable epidermis across the root sheath. The co-emulsifier may be a part of the lipophilic or of the hydrophilic phase of the emulsion, but is preferably a component of the hydrophilic phase. The co-emulsifier may be ionic, non-ionic, or zwitterionic. Preferred co-emulsifiers are non-ionic and include one or more of polysorbates, such as polysorbate 40, 60, or 80, poloxamers, such as those that are sold under the trade name PLURONIC® (BASF, Washington, N.J.), laureths, ceteths, and steareths, such as those sold under the trade name BRIJ® (ICI America of Wilmington, Del.). The concentration of the co-emulsifier in the emulsion of the invention is from 0% to 10% or higher, preferably from 2% to 8%, and most preferably from 4% to 6% w/w.

The formulation further optionally contains additional excipients used in topical pharmaceutical formulations including but not limited to preservatives such as benzalkonium chloride, benzethonium, chlorhexidine, phenol, m-cresol, benzyl alcohol, methylparaben, propylparaben, chlorobutanol, o-cresol, p-cresol, chlorocresol, phenylmercuric nitrate, thimerosal, and benzoic acid; buffers; emollients such as cetyl alcohol, isopropyl myristate, stearyl alcohol; thickening agents such as a cellulose-based thickener like hydroxyethylcellulose or hydroxypropylcellulose or carbomer homopolymer thickeners including Carbopol® 934, 940, 941, 980, or 981 (Noveon, Inc., Akron, Ohio, USA); or anti-oxidants such as butylated hydroxytoluene, ascorbic acid, sodium ascorbate, calcium ascorbate, ascorbic palmitate, butylated hydroxyanisole, 2,4,5-trihydroxybutyrophenone, 4-hydroxymethyl-2,6-di-tert-butylphenol, erythorbic acid, gum guaiac, propyl gallate, thiodipropionic acid, dilauryl thiodipropionate, tert-butylhydroquinone and tocopherols such as vitamin E.

The invention is further illustrated by the following non-limiting examples.

### EXAMPLE 1

### Prior Art

A positive control enhanced delivery solution of an API was made by mixing, in the following proportions, 20% water, 19% propylene glycol, and 60% ethanol, and 1% verapamil as the hydrochloride. All of the verapamil was dissolved in the enhanced delivery solution. The enhanced delivery system was designated “Formulation 1.1”.

### EXAMPLE 2

### Prior Art

A cholate/lecithin solution, designated “Formulation 1.2” was made by mixing, in the following proportions, 74% water, 15% sodium cholate, 10% lecithin soya granules and 1% verapamil as the hydrochloride. A cholate/lecithin gel, designated “Formulation 1.3” was made by mixing, in the following proportions, 73% water, 15% sodium cholate, 10% lecithin soya granules, 1% poly(ethyleneoxide), and 1% verapamil. All of the verapamil HCl was dissolved in each of the Formulations 1.2 and 1.3.

### EXAMPLE 3

A water-in-silicone emulsion of the invention, designated “Formulation 1.4”, was made as follows. A hydrophilic phase of the drug verapamil (solubility of verapamil hydrochloride is 7 g/100 g water) was made by dissolving 1 gram of verapamil as the hydrochloride in 24 grams of water combined with 5 grams of propylene glycol and 2 grams of polysorbate 20. This hydrophilic solvent phase was mixed until the API verapamil was completely dissolved. In a separate container, a lipophilic silicone phase was made by combining 20 grams of Dow Coming® 345 Cyclomethicone Fluid (decamethylcyclopentasiloxane) with 30 grams of Dow Coming® 344 Cyclomethicone Fluid (octamethylcyclotetrasiloxane), 8 grams of Dow Corning® 3225C Formulation Aid (a blend of a silicone emulsifier in Dow Coming® 344 Fluid), 5 grams of Dow Coming ST Cyclomthicone 5-NF Fluid (decamethylcyclopentasiloxane), and 5 grams of oleyl alcohol. After complete mixing of the silicone phase, the hydrophilic phase was added to the silicone phase with high shear mixing to form a water-in-silicone emulsion cream.

### EXAMPLE 4

A water-in-silicone emulsion of the invention, designated “Formulation 1.5”, was made as follows. A hydrophilic phase of the drug verapamil was made by dissolving 1 gram of verapamil as the hydrochloride in 24 grams of water combined with 5 grams of propylene glycol and 2 grams of polysorbate 20. This hydrophilic solvent phase was mixed until the API verapamil was completely dissolved. In a separate container, a lipophilic silicone phase was made by combining 15 grams of Dow Corning® 345 Cyclomethicone Fluid (decamethylcyclopentasiloxane) with 25 grams of Dow Corning® 344 Cyclomethicone Fluid (octamethylcyclotetrasiloxane), 8 grams of Dow Coming® 3225C Formulation Aid (a blend of a silicone emulsifier in Dow Coming® 344 Fluid), 10 grams of Dow Coming® 200 Dimethicone Fluid (polydimethylsiloxane), 5 grams of Dow Coming ST Cyclomthicone 5-NF Fluid (decamethylcyclopentasiloxane), and 5 grams of oleyl alcohol. After complete mixing of the silicone phase, the hydrophilic phase was added to the silicone phase with high shear mixing to form a water-in-silicone emulsion cream.

### EXAMPLE 5

Formulation 1.1 of Example 1, Formulation 1.2 of Example 2, and Formulation 1.4 of Example 3 were each spiked with tracer amounts of radiolabeled (³H)-verapamil as the hydrochloride at approximately 0.90 μCi/dose. A single clinically relevant dose (5 mg/cm²) was applied to dermatomed human skin obtained from one donor following elective surgery.

Percutaneous absorption was evaluated by mounting the dermatomed tissue in Bronaugh flow-through diffusion cells at 32° C. Six replicates were performed for each formulation. Fresh receptor fluid, PBS containing 0.1% w/v sodium azide and 4% w/v Bovine Serum Albumin, was continuously pumped under the skin at a nominal flow rate of 1 ml/hr and collected in 6-hour intervals. Following 24-hours of exposure, the residual formulation remaining on the skin surface was removed by repeated tape stripping (5 strips/cell). Subsequently, the epidermis was physically separated from the dermis by gentle peeling. The quantity of radioactivity in the tape-strips, epidermis, dermis, and receptor fluid samples was determined using liquid scintillation counting.

TABLE 1

| | % Dose applied (Mean) | | | | | |
|---|---|---|---|---|---|---|
| | | Tape | Tape | | | |
| | Tape | strips | strips | | | Re- |
| | strip 1 | 2–3 | 4–5 | Epidermis | Dermis | ceptor |
| Formulation 1.1 | 12.04 | 12.87 | 7.94 | 38.23 | 7.56 | 0.563 |
| Formulation 1.2 | 36.59 | 14.57 | 3.71 | 17.34 | 2.92 | 0.227 |
| Formulation 1.3 | 61.43 | 9.11 | 2.77 | 11.17 | 0.69 | 0.133 |
| Formulation 1.4 | 25.29 | 16.61 | 7.84 | 28.54 | 3.61 | 0.574 |
| Formulation 1.5 | 27.44 | 13.49 | 6.31 | 41.15 | 1.23 | 0.438 |

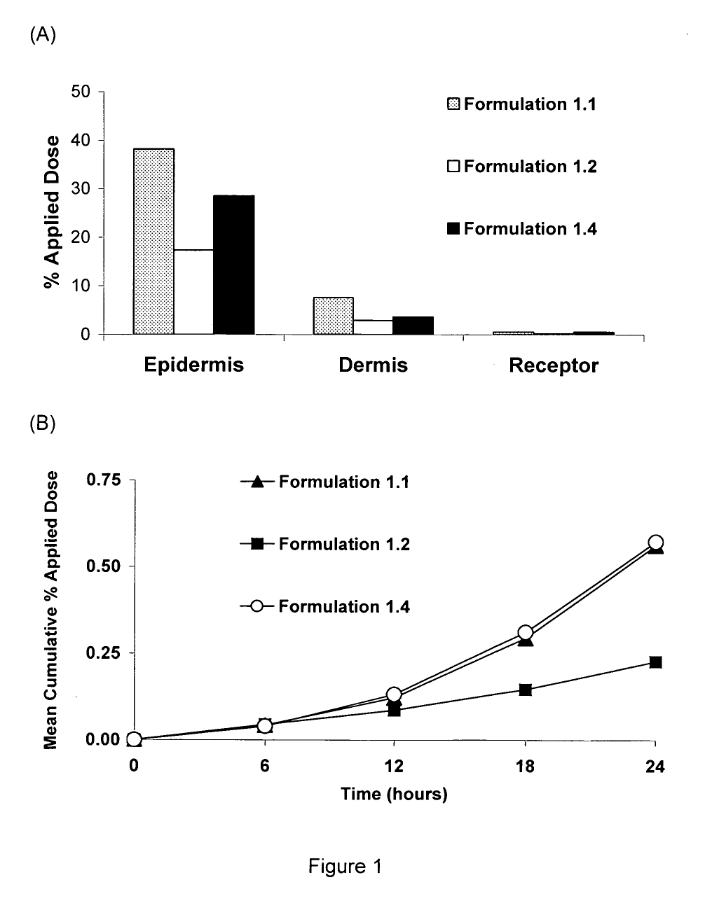

Data is shown in Table 1 and FIG. 1. The cholate/lecithin solution of Formulation 1.2 demonstrated low levels of percutaneous absorption and epidermal deposition of the API verapamil. In contrast, percutaneous absorption and epidermal deposition of the API verapamil was statistically identical from both the silicone-containing formulation of the invention, Formulation 1.4, and from the enhanced delivery solution, Formulation 1.1.

Formulation 1.1 is a positive control for skin penetration. It contains high levels of the skin penetration enhancers ethanol (60%) and propylene glycol (19%) that tend to dry and irritate the skin. By formulating the verapamil in a water-in-silicone emulsion of the invention, similar levels of skin penetration enhancement and dermal deposition are obtained while permitting the reduction of the irritants propylene glycol to 5% and ethanol to 0%. The irritation potential of propylene glycol and ethanol is significantly reduced when these excipients are used at 10% or less in a topical formulation. The formulation of the invention, which has far superior aesthetic, cosmetic, and emolliency characteristics, provides a more cosmetically acceptable alternative to ethanolic formulations while maintaining the ability to promote skin penetration and dermal deposition of the API.

### EXAMPLE 6

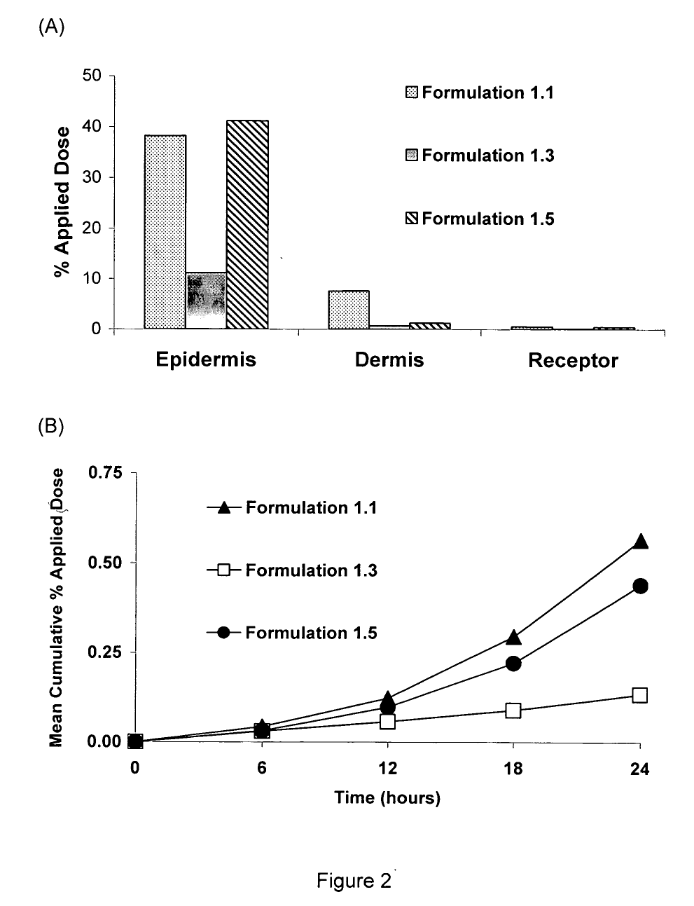

The procedure of Example 5 was repeated utilizing Formulation 1.1 of Example 1, Formulation 1.3 of Example 2, and Formulation 1.5 of Example 4. Data is shown in Table 1 and FIG. 2. The cholate/lecithin solution of Formulation 1.3 demonstrated low levels of percutaneous absorption and epidermal deposition of the API verapamil. In contrast, percutaneous absorption and epidermal deposition of the API verapamil was statistically identical from both the silicone-containing formulation of the invention, Formulation 1.5, and from the enhanced delivery solution, Formulation 1.1.

### EXAMPLE 7

A water in silicone emulsion cream, designated “Formulation 2.9”, of a 12-15 kDa oligonucleotide (solubility of oligonucleotide is greater than 10 g/100 g water) was made by dissolving 0.5 gram of oligonucleotide in 39.3 grams of water combined with 5 grams of propylene glycol, 5 grams of polysorbate 20, 0.03 grams of propylparaben, 0.17 grams of methylparaben and 10 grams of 100 mM phosphate buffer (1.5M NaCl). This hydrophilic solvent phase was mixed until the oligonucleotide API was completely dissolved. In a separate container, 31 grams of Dow Corning® ST Cyclomethicone 5-NF Fluid, 8 grams of Dow Corning® 3225C Formulation Aid, and 1 gram of Dow Corning® Q7 9120 Dimethicone (100 cS) (polydismethylsiloxane). After complete mixing of the silicone phase, the hydrophilic phase was steadily added to the silicone phase with high shear mixing to form the water-in-silicone emulsion cream.

### EXAMPLE 8

### Prior Art

An oil-in-water emulsion, designated “Formulation 2.5”, was made by combining 12 grams of caprylic/capric triglyceride as an internal phase, and an aqueous internal phase containing 10 grams of hydrogenated phosphatidylcholine, 3 grams of Laureth-4, 10 grams of dehydrated ethanol, 10 grams of 100 mM phosphate buffer (1.5 M NaCl), 0.17 grams methylparaben, 0.3 grams propylparaben, 54.3 grams water and 0.5% of the oligonucleotide (non-radiolabeled).

An aqueous based gel, designated “Formulation 2.7”, was made by blending 5 grams of dehydrated ethanol, 10 grams of 100 mM phosphate buffer (1.5 M NaCl), 0.17 grams of methylparaben, 0.3 grams of propylparaben, 3 grams of nonoxynol-9, 1.3 grams of hydroxyethyl cellulose, 75 grams of water and 0.5% of the oligonucleotide (non-radiolabeled).

### EXAMPLE 9

Each of the Formulations 2.5, 2.7, and 2.9 of Examples 7 and 8 was spiked with tracer amounts of the radiolabeled 12-15 kDa oligonucleotide at approximately 1.0 μCi/dose. A single clinically relevant dose (5 mg/cm²) was applied to dermatomed human skin obtained from one donor following elective surgery.

Percutaneous absorption was evaluated by mounting the dermatomed tissue in Bronaugh flow-through diffusion cells at 32° C. Six replicates were performed for each formulation. Fresh receptor fluid, PBS containing 0.1% w/v sodium azide and 4% w/v Bovine Serum Albumin, was continuously pumped under the skin at a nominal flow rate of 1 ml/hr and collected in 6-hour intervals. Following 24-hours of exposure, the residual formulation remaining on the skin surface was removed by repeated tape stripping (5 strips/skin sample). Subsequently, the epidermis was physically separated from the dermis by gentle peeling. The quantity of radioactivity in the tape-strips, epidermis, dermis, and receptor fluid samples was determined using liquid scintillation counting.

TABLE 2

| | Tape | | | |
|---|---|---|---|---|
| | strips | Epidermis | Dermis | Receptor |
| Formulation 2.5 | 91.00 | 1.19 | 0.07 | 0.039 |
| Formulation 2.7 | 96.79 | 0.37 | 0.02 | 0.031 |
| Formulation 2.9 | 72.34 | 10.70 | 0.39 | 0.026 |

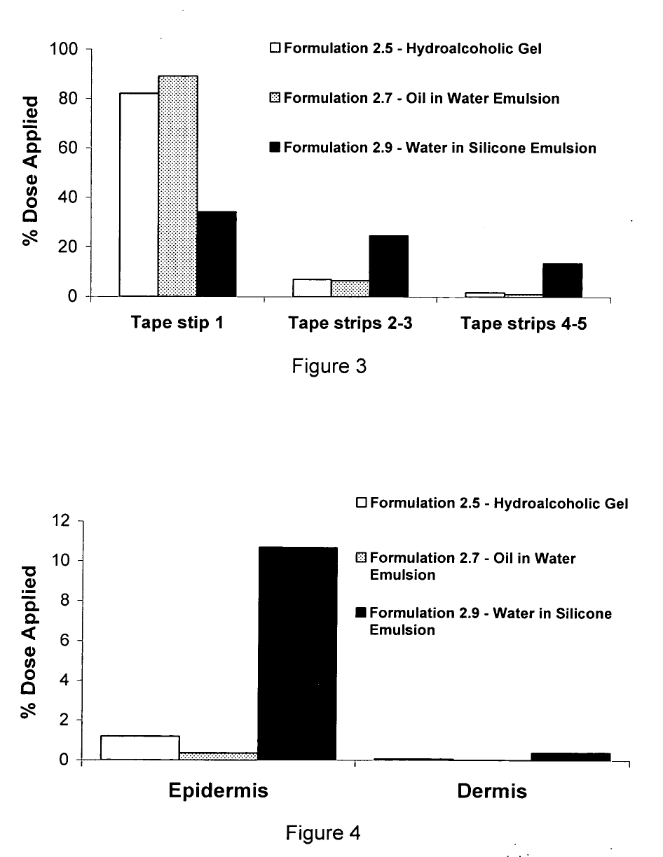

The results are shown in Table 2 and in FIGS. 3 and 4. As shown in FIG. 3, the greater than 90% of the applied dose from the hydroalcoholic gel (Formulation 2.5) and the oil-in-water emulsion (Formulation 2.7) were recovered in the tape strips, indicating that almost all of the applied oligonucleotide from these formulations remained on the skin surface. In contrast, only about 72% of the oligonucleotide from the water-in-silicone emulsion crearn (Formulation 2.9) was recovered from the tape strips, indicating that a much higher percentage of the oligonucleotide from this formulation penetrated into the skin.

As shown in FIG. 4, statistically higher (p≦0.027) levels of oligonucleotide from the silicone-containing Formulation 2.9 was also observed in the epidermis and dermis compared with the other two formulations. All formulations resulted in similar, very low levels of the oligonucleotide in the receptor solution, i.e. less than 0.05% of the applied dose. The data shows dramatically increased deposition into the dermis and epidermis of the 12-15 kDa oligonucleotide when applied as a water-in silicone emulsion in accordance with the invention compared to the prior art formulations.

### EXAMPLE 10

Three water-in-silicone emulsion cream formulations containing a macrolide antibiotic (solubility of the macrolide is greater than 0.1 g/100 g hydrophilic solvent phase) were made and were designated Formulations 1432:94, 1432:95, and 1432:96, respectively. The hydrophilic solvent phases of the formulations were made by dissolving 0.1 gram of the macrolide in 57.7 grams (Formulation 1432.94), 47.7 grams (Formulation 1432:95), or 57.2 grams (Formulation 1432:96) of citrate buffer (100 mM, pH 5.5) combined with 5 grams of propylene glycol, 5 grams of polysorbate 20, 2 grams of sodium chloride, 0.03 grams of propylparaben, and 0.17 grams of methylparaben. In Formulation 1432:95, 0.5 grams of PEG 400 monolaurate was also combined in the hydrophilic solvent phase. The respective hydrophilic solvent phases were mixed until the macrolide API was completely dissolved.

In separate containers, the lipophilic silicone phases for the formulations were made as follows. For Formulations 1432:94 and 1432:96, 21 grams of Dow Corning ST Cyclomethicone 5-NF Fluid, 8 grams of Dow Corning 3225C Formulation Aid, and 1 gram of Dow Coming Q7 9120 Dimethicone (100 cS) were combined. The primary component of Dow Coming Q7 9120 Dimethicone (100 cS) fluid is polydismethylsiloxane. For Formulation 1432:95, 27 grams of Dow Coming ST Cyclomethicone 5-NF Fluid, 8 grams of Dow Coming 3225C Formulation Aid, 1 gram of Dow Corning Q7 9120 Dimethicone (100 cS), and 4 grams of ST-Elastomer 10 were combined. Dow Coming ST-Elastomer 10 is composed of 12% silicone elastomer in decylmethylcyclopentasiloxane.

After complete mixing of the respective silicone phases, the respective hydrophilic phase was steadily added to the silicone phase with high shear mixing to form the water-in-silicone emulsion creams. The compositions of the silicone-containing macrolide emulsions 1432:94, 1432:95, and 1432:96 are shown in Table 3.

TABLE 3

| | Formulation No. | | |
|---|---|---|---|
| Components | 1432:94 | 1432:95 | 1432:96 |
| Macrolide | 0.10 | 0.10 | 0.10 |
| Propylene glycol (USP) | 5.00 | 5.00 | 5.00 |
| Methylparaben (NF) | 0.17 | 0.17 | 0.17 |
| Propylparaben (NF) | 0.03 | 0.03 | 0.03 |
| Citrate Buffer | 57.70 | 47.70 | 57.20 |
| Polysorbate 20 (Tween 20, NF) | 5.00 | 5.00 | 5.00 |
| PEG 400 monolaurate | — | — | 0.50 |
| Sodium Chloride | 2.00 | 2.00 | 2.00 |
| 3225C Formulation Aid | 8.00 | 8.00 | 8.00 |
| ST-Cyclomethicone (NF) | 21.00 | 27.00 | 21.00 |
| Q7 9120 Dimethicone (100 CS) | 1.00 | 1.00 | 1.00 |
| St-Elastomer 10 | — | 4.00 | — |
| Total | 100.00 | 100.00 | 100.00 |
| % Hydrophilic phase | 70.00 | 60.00 | 70.00 |
| % Silicone phase | 30.00 | 40.00 | 30.00 |

### EXAMPLE 11

Eight test formulations of the invention, designated 2227-60, 2227-65, 2227-70, 2227-71, 2227-78, 2227-84, 2227-85, and 2227-91 were made by the method described in Example 7. Briefly, hydrophilic components were mixed to form a hydrophilic solvent phase. In a separate container, silicone and hydrophobic components were mixed to form a silicone phase. The hydrophilic solvent phase was steadily added to the silicone phase with high shear mixing to form a water-in-silicone emulsion cream. Two control formulations, a 100% silicone phase formulation designated 2227-32 and a water-in-hydrocarbon oil emulsion, lacking any silicone component in the oil phase, designated 2227-33A-34, were also made. The compositions of the formulations of the invention and the control formulations are shown in Table 4.

TABLE 4

| | Formulation ID | | | | | | | | | |
|---|---|---|---|---|---|---|---|---|---|---|
| | | | | | | | | | | 2227- |
| | 2227- | 2227- | 2227- | 2227- | 2227- | 2227- | 2227- | 2227- | 2227- | 33A- |
| | 60 | 65 | 70 | 71 | 78 | 84 | 85 | 91 | 32 | 34 |
| Components | % w/w | | | | | | | | | |
| Propylene glycol | 5.00 | 5.00 | 5.00 | — | 5.0 | — | — | 4.00 | — | — |
| (USP) | | | | | | | | | | |
| PEG-22/Dodecyl | — | — | — | — | — | — | — | — | — | 3.0 |
| Glycol Copolymer | | | | | | | | | | |
| Methylparaben (NF) | 0.17 | 0.17 | 0.17 | 0.17 | 0.17 | 0.17 | 0.17 | 0.17 | — | — |
| Propylparaben (NF) | 0.03 | 0.03 | 0.03 | 0.03 | 0.03 | 0.03 | 0.03 | 0.03 | — | — |
| Purified water (USP) | 47.80 | 48.30 | 47.80 | 57.80 | 49.30 | 28.80 | 38.30 | 65.30 | — | 62.5 |
| Polysorbate 20 (Tween | 5.00 | 5.00 | 5.00 | 5.00 | 5.00 | 5.00 | 5.00 | — | — | — |
| 20, NF) | | | | | | | | | | |
| Transcutol | — | — | — | 5.00 | — | 5.00 | 5.00 | — | — | — |
| (ethoxydiglycol) | | | | | | | | | | |
| Sodium Chloride | 2.00 | — | 2.00 | 2.00 | — | — | — | — | — | — |
| Magnesium Chloride | — | 0.50 | — | — | 0.50 | 1.00 | 0.50 | 0.50 | — | — |
| Magnesium Sulfate | — | — | — | — | — | — | — | — | — | 0.5 |
| Sorbitan Monooleate | — | — | — | — | — | — | — | — | — | 3.0 |
| White Wax | — | — | — | — | — | — | — | — | — | 5.0 |
| Mineral Oil | — | — | — | — | — | — | — | — | — | 20.0 |
| White Petrolatum | — | — | — | — | — | — | — | — | — | 6.0 |
| Brij 36T | — | — | — | — | — | — | 1.00 | — | — | — |
| 3225C Formulation | 8.00 | 8.00 | 8.00 | 8.00 | 8.00 | 8.00 | 8.00 | — | 20.0 | — |
| Aid (Cyclomethicone | | | | | | | | | | |
| and PEG/PPG-18/18 | | | | | | | | | | |
| Dimethicone) | | | | | | | | | | |
| Toray FZ-2233 | — | — | — | — | — | — | — | 2.00 | — | — |
| (Polysilicone 13, Dow | | | | | | | | | | |
| Corning Corp.) | | | | | | | | | | |
| ST-Cyclomethicone | 31.00 | 31.00 | 26.00 | 21.00 | 27.00 | 27.00 | 27.00 | 22.50 | 77.5 | — |
| (NF) | | | | | | | | | | |
| Q7 9120 Dimethicone | 1.00 | 1.00 | 1.00 | 1.00 | — | 10.00 | 10.00 | 5.00 | 2.5 | — |
| (100 CS) | | | | | | | | | | |
| Q7 9120 Dimethicone | — | — | — | — | 5.00 | — | — | — | — | — |
| (350 CS) | | | | | | | | | | |
| Capric/Caprylic | — | — | — | — | — | 5.00 | 5.00 | — | — | — |
| Triglycerides | | | | | | | | | | |
| Dimethicone PEG-7 | — | 1.00 | — | — | — | — | — | — | — | — |
| Cocoate | | | | | | | | | | |
| Oleth-2 (Brij 93) | — | — | 5.00 | — | — | — | — | — | — | — |
| Elastomer 10 (silicone | — | — | — | — | — | 10.00 | — | — | — | — |
| elastomer gel) | | | | | | | | | | |
| PPG 3 Myristyl Ether | — | — | — | — | — | — | — | 0.50 | — | — |
| Total (%) | 100 | 100 | 100 | 100 | 100 | 100 | 100 | 100 | 100 | 100 |

### EXAMPLE 12

¹⁴C-caffeine was selected as a model permeant to evaluate the topical delivery efficiency mediated by the formulations of Example 11. Caffeine is classified as a compound having relatively high skin permeability. It has a molecular weight of 194.19, log P −0.07, and an aqueous solubility of 21.6 g/L.

The formulations of Example 11 were spiked with sufficient radio-tracer (¹⁴C-caffeine) to achieve a nominal 0.5 μCi dose per diffusion cell at a topical application of 5 mg formulation per square centimeter of tissue. The radiolabeled compounds were incorporated at tracer levels to eliminate the effect of thermodynamic activity (degree of permeant saturated solubility in the formulations and residual). This single, clinically relevant dose was applied to dermatomed human abdominal skin from a single donor obtained following elective surgery. Five replicates were performed for each formulation. The thickness of the tissue ranged from 0.610 mm to 0.914 mm with a mean±standard deviation in thickness of 0.773±0.072 mm and a coefficient of variation of 9.35%.

Percutaneous absorption was evaluated using this human abdominal tissue which was mounted in Bronaugh flow-through diffusion cells. The cells were maintained at a constant temperature of 32° C. by use of recirculating water baths. The cells have a nominal diffusion area of 0.64 cm². Fresh receptor fluid, PBS with 0.1% sodium azide and 1.5% Oleth-20, was continuously pumped under the tissue at a flow rate of nominally 1 ml/hr and collected in 6-hour intervals. Following a 24-hour exposure period, the formulation residing on the skin was removed by tape-stripping up to five consecutive times with D-Squame® stripping discs (CuDerm Corporation, Dallas, Tex.). Delivery efficiency was measured as total % of the applied dose recovered from the epidermis and dermis of the excised human tissue.

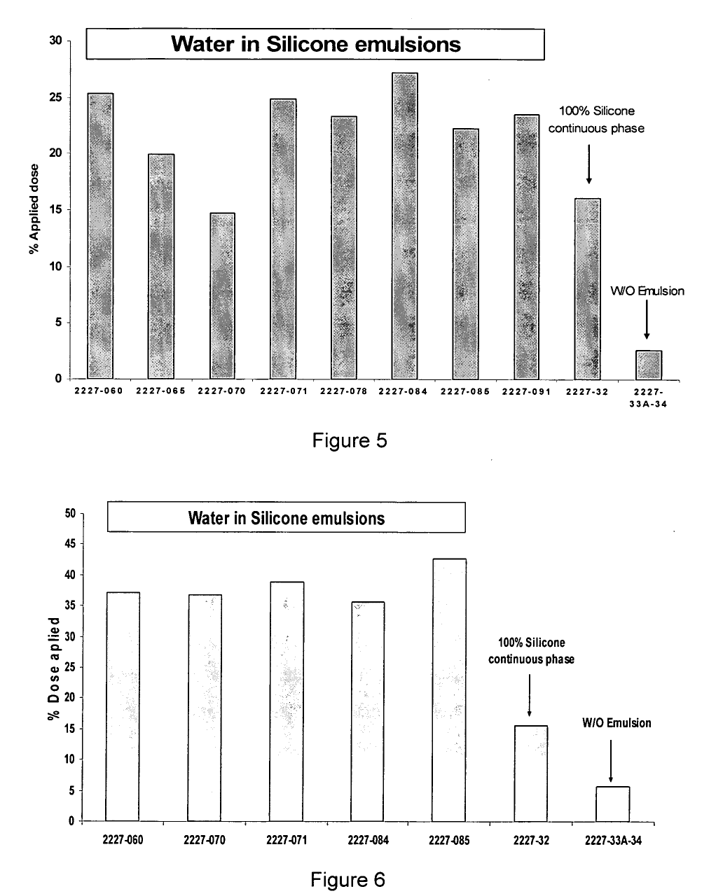

Data from this study is summarized in Table 5 and in FIG. 5. As shown in Table 5 and FIG. 5, the water-in-silicone emulsions of the invention generated 5.6 to 10.5 times greater tissue deposition of the caffeine tracer than did the control water-in-oil emulsion (statistically significantly different p<0.05) and all but one of the water-in-silicone emulsions generated greater tissue deposition of the caffeine tracer than did the silicone continuous phase. The water-in-hydrocarbon oil control emulsion provided the greatest amount of caffeine that penetrated the skin, but skin deposition from this emulsion was very low.

TABLE 5

| Formulation | | Percent of Applied Dose | | | |
|---|---|---|---|---|---|
| ID | | Epidermis | Dermis | Total Skin | Receptor Phase |
| 2227-060 | Mean | 20.34 | 5.00 | 25.34 | 53.49 |
| | SD | 7.6 | 2.5 | 6.7 | 12.91 |
| | % RSD | 37.4 | 50.1 | 26.3 | 24.1 |
| 2227-065 | Mean | 11.05 | 8.90 | 19.95 | 46.08 |
| | SD | 4.7 | 8.5 | 7.8 | 14.18 |
| | % RSD | 42.6 | 95.3 | 38.9 | 30.8 |
| 2227-070 | Mean | 9.64 | 5.04 | 14.68 | 66.69 |
| | SD | 5.8 | 2.3 | 6.8 | 14.67 |
| | % RSD | 59.9 | 46.3 | 46.6 | 22.0 |
| 2227-071 | Mean | 21.42 | 3.41 | 24.83 | 35.85 |
| | SD | 7.4 | 1.2 | 7.3 | 11.69 |
| | % RSD | 34.3 | 35.3 | 29.3 | 32.6 |
| 2227-078 | Mean | 21.42 | 1.94 | 23.36 | 39.81 |
| | SD | 4.1 | 1.0 | 4.4 | 12.19 |
| | % RSD | 19.0 | 50.8 | 19.0 | 30.6 |
| 2227-084 | Mean | 22.99 | 4.20 | 27.20 | 40.58 |
| | SD | 4.1 | 0.7 | 3.9 | 14.22 |
| | % RSD | 17.9 | 16.7 | 14.3 | 35.1 |
| 2227-085 | Mean | 16.12 | 6.15 | 22.28 | 65.53 |
| | SD | 4.3 | 1.2 | 5.4 | 10.52 |
| | % RSD | 26.5 | 19.9 | 24.4 | 16.1 |
| 2227-091 | Mean | 21.57 | 1.94 | 23.51 | 42.67 |
| | SD | 8.2 | 1.2 | 8.5 | 14.23 |
| | % RSD | 38.2 | 63.1 | 36.1 | 33.4 |
| 2227-32 | Mean | 10.91 | 5.14 | 16.05 | 55.40 |
| | SD | 4.6 | 1.4 | 5.3 | 7.17 |
| | % RSD | 42.4 | 27.0 | 33.3 | 12.9 |
| 2227-33A-34 | Mean | 1.47 | 1.17 | 2.64 | 80.99 |
| | SD | 0.7 | 0.3 | 0.7 | 4.87 |
| | % RSD | 45.1 | 22.9 | 27.6 | 6.0 |

### EXAMPLE 13

The study of Example 12 was repeated except that ¹⁴C-glucose was utilized as the model permeant to evaluate the topical delivery efficiency mediated by the formulations of Example 11 and only the test formulations of the invention 2227-60, 2227-70, 2227-71, 2227-84, and 2227-85, as well as the two control formulations, were evaluated. Glucose is classified as a compound having poor skin permeability. It has a molecular weight of 180.16, log P −3.24, and an aqueous solubility of 1200 g/L.

Data from this study is summarized in Table 6 and in FIG. 6. As shown in Table 6 and FIG. 6, the water-in-silicone emulsions of the invention generated 6.3 to 7.5 times greater tissue deposition of the caffeine tracer than did the control water-in-oil emulsion (statistically significantly different p<0.05) and all but one of the water-in-silicone emulsions generated greater tissue deposition of the caffeine tracer than did the silicone continuous phase. In contrast to the caffeine study of Example 12, the water-in-hydrocarbon oil control emulsion provided the lowest amount of glucose that penetrated the skin.

TABLE 6

| Formulation | Percent of Applied Dose | | | | |
|---|---|---|---|---|---|
| ID | | Epidermis | Dermis | Total Skin | Receptor Phase |
| 2227-060 | Mean | 32.56 | 4.53 | 37.10 | 0.16 |
| | SD | 10.5 | 2.3 | 8.6 | 0.02 |
| | % RSD | 32.4 | 51.3 | 23.1 | 14.1 |
| 2227-070 | Mean | 32.00 | 4.77 | 36.77 | 0.54 |
| | SD | 16.4 | 4.4 | 18.4 | 0.23 |
| | % RSD | 51.1 | 91.8 | 50.0 | 42.4 |
| 2227-071 | Mean | 33.82 | 5.00 | 38.82 | 0.24 |
| | SD | 2.5 | 2.8 | 3.9 | 0.17 |
| | % RSD | 7.5 | 55.7 | 10.0 | 74.1 |
| 2227-084 | Mean | 31.60 | 4.11 | 35.71 | 0.17 |
| | SD | 12.4 | 2.8 | 14.7 | 0.09 |
| | % RSD | 39.2 | 69.2 | 41.2 | 51.0 |
| 2227-085 | Mean | 38.69 | 3.99 | 42.68 | 0.34 |
| | SD | 9.9 | 2.1 | 10.5 | 0.21 |
| | % RSD | 25.5 | 51.9 | 24.5 | 62.8 |
| 2227-32 | Mean | 12.71 | 2.96 | 15.67 | 0.18 |
| | SD | 7.5 | 2.6 | 7.8 | 0.18 |
| | % RSD | 58.9 | 86.9 | 49.9 | 99.2 |
| 2227-33A-34 | Mean | 5.39 | 0.35 | 5.74 | 0.05 |
| | SD | 5.2 | 0.2 | 5.3 | 0.02 |
| | % RSD | 95.6 | 59.8 | 92.3 | 49.5 |

Further modifications, uses, and applications of the invention described herein will be apparent to those skilled in the art. It is intended that such modifications be encompassed in the following claims.

1. A stable emulsion formulation for topical administration to the skin of a mammal, comprising an internal hydrophilic phase containing a hydrophilic solvent, a pharmaceutically active compound dissolved or dispersed in whole or in part in the hydrophilic solvent, a continuous lipophilic phase containing a silicone oil, and an emulsifier dissolved in whole or in part in the continuous phase.

2. The emulsion formulation of claim 1 wherein the hydrophilic solvent is water.

3. The emulsion formulation of claim 1 wherein the hydrophilic solvent is other than water.

4. The emulsion formulation of claim 3 wherein the hydrophilic solvent is miscible with water.

5. The emulsion formulation of claim 1 wherein the pharmaceutically active compound is hydrophilic and is dissolved essentially exclusively in the hydrophilic phase.

6. The emulsion formulation of claim 1 wherein the pharmaceutically active compound is partitioned between the hydrophilic and silicone phases.

7. The emulsion formulation of claim 1 wherein the pharmaceutically active compound is dispersed within the hydrophilic phase.

8. The emulsion formulation of claim 1 which further comprises a co-emulsifier.

9. The emulsion formulation of claim 1 wherein the silicone oil is one or more volatile silicones.

10. The emulsion formulation of claim 1 wherein the silicone oil is one or more non-volatile silicones.

11. The emulsion formulation of claim 1 wherein the silicone oil is a combination of one or more volatile silicones and one or more non-volatile silicones.

12. The emulsion formulation of claim 1 wherein the silicone oil is the major component of the continuous phase.

13. The emulsion formulation of claim 1 wherein the emulsifier is a silicone-based surfactant.

14. A method for making an emulsion formulation for topical administration to the skin of a mammal comprising combining a hydrophilic solvent, a lipophilic phase containing a silicone oil, and an emulsifier, wherein the emulsifier is soluble in the lipophilic phase, to produce a stable water-in-oil emulsion, and dissolving or dispersing a pharmaceutically active compound in the hydrophilic solvent.

15. The method of claim 14 wherein the pharmaceutically active compound is dissolved or dispersed in the hydrophilic solvent before the hydrophilic solvent is combined with the lipophilic phase.

16. The method of claim 14 wherein the hydrophilic solvent is water.

17. The method of claim 14 wherein the hydrophilic solvent is other than water.

18. The method of claim 17 wherein the hydrophilic solvent is miscible with water.

19. The method of claim 14 wherein the pharmaceutically active compound is hydrophilic and dissolves completely in the hydrophilic phase.

20. The method of claim 14 wherein the pharmaceutically active compound is hydrophobic and is dispersed in the hydrophilic phase.

21. The method of claim 14 which further comprises combining a co-emulsifier that is soluble in either or both of the hydrophilic solvent and the lipophilic phase.

22. The method of claim 14 wherein the silicone oil is one or more volatile silicones.

23. The method of claim 14 wherein the silicone oil is one or more non-volatile silicones.

24. The method of claim 14 wherein the silicone oil is a combination of one or more volatile silicones and one or more non-volatile silicones.

25. The method of claim 14 wherein the silicone oil is the major component of the lipophilic phase.

26. The method of claim 14 wherein the emulsifier is a silicone-based surfactant.

27. A method for increasing the penetration of a pharmaceutically active chemical compound through the stratum corneum of mammalian skin or for increasing the deposition of a chemical compound in human skin comprising topically administering to the skin of a mammalian subject in need thereof a stable water-in-oil emulsion formulation comprising the pharmaceutically active chemical compound and further comprising an internal hydrophilic phase containing a hydrophilic solvent in which the pharmaceutically active compound is partially or completely dissolved or dispersed, a continuous lipophilic phase containing a silicone oil, and an emulsifier dissolved in whole or in part in the continuous phase.

28. The method of claim 27 wherein the hydrophilic solvent is water.

29. The method of claim 27 wherein the hydrophilic solvent is other than water.

30. The method of claim 29 wherein the hydrophilic solvent is miscible with water.

31. The method of claim 27 wherein the pharmaceutically active compound is hydrophilic and dissolves completely in the hydrophilic phase.

32. The method of claim 27 wherein the pharmaceutically active compound is hydrophobic and is dispersed in the hydrophilic phase.

33. The method of claim 27 wherein the emulsion formulation further comprises a co-emulsifier that is dissolved in either or both of the hydrophilic solvent and the lipophilic phase.

34. The method of claim 27 wherein the silicone oil is one or more volatile silicones.

35. The method of claim 27 wherein the silicone oil is one or more non-volatile silicones.

36. The method of claim 27 wherein the silicone oil is a combination of one or more volatile silicones and one or more non-volatile silicones.

37. The method of claim 27 wherein the silicone oil is the major component of the lipophilic phase.

38. The method of claim 27 wherein the emulsifier is a silicone-based surfactant.
